(19) United States
(12) Patent Application Publication
(10) Pub. No.: US 2025/0239149 A1
(43) Pub. Date: Jul. 24, 2025

(54) SYSTEM AND METHOD FOR REMOTE MONITORING OF INDIVIDUALS

(71) Applicant: AGENT MONITORING, LLC, Houston, TX (US)

(72) Inventor: Melissa Montes, Houston, TX (US)

(73) Assignee: AGENT MONITORING, LLC, Houston, TX (US)

(21) Appl. No.: 19/032,195

(22) Filed: Jan. 20, 2025

Related U.S. Application Data
(60) Provisional application No. 63/622,651, filed on Jan. 19, 2024.

(51) Int. Cl. G08B 25/10; A61B 5/00; A61B 5/08; G06T 7/00; G06V 40/16; G08B 29/04; H04M 1/72412; H04N 23/63

(52) U.S. Cl. CPC G08B 25/10; A61B 5/082; A61B 5/4845; G06T 7/0016; G06V 40/172; G08B 29/046; H04M 1/72412; H04N 23/63; G06T 2207/10016; G06T 2207/30004; G06T 2207/30201

(57) ABSTRACT

Embodiments of a method to test a user for a substance include recording a video from a camera via the personal mobile device and capturing a first photograph from the camera via the personal mobile device. While the video is being recorded by the personal mobile device, the method further includes measuring a blood alcohol content of the user via the portable testing device. After measuring the blood alcohol content, the method includes capturing a second photograph from the camera via the personal mobile device and comparing the first photograph and the second photograph via a facial recognition routine. Further, the method includes analyzing the blood alcohol content of the user compared to a threshold blood alcohol content.

### RELATED APPLICATIONS

This application claims the benefit of priority U.S. Provisional Patent Application Ser. No. 63/622,651 entitled “SYSTEM AND METHOD FOR REMOTE MONITORING OF INDIVIDUALS” filed on Jan. 19, 2024, the disclosure of which is hereby incorporated by reference in its entirety for all purposes.

### FIELD

The present disclosure generally relates to remote monitoring of individuals, and more particularly, to remote sobriety monitoring of individuals.

### BACKGROUND

Monitoring systems can allow probation officers or other agents to monitor the activities, behaviors, and/or actions of individuals as part of a probation or conditional release program. Monitoring systems can include components that record information regarding the target individual, including, but not limited to blood alcohol content (BAC) of the target individual, store and/or transmit information regarding the target individual to the monitoring agent, and restrict or interlock the operation of a motor vehicle.

Certain conventional monitoring systems may be defeated or bypassed by the target individual. For example, a target individual may utilize a device or third party to perform a required breathalyzer test. Further, the target individual may intentionally or unintentionally miss a scheduled breathalyzer test. In another example, a target individual may bypass a vehicle interlock system by using a different vehicle without the required interlock device. Further, certain conventional monitoring systems may not monitor for drug use, such as tetrahydrocannabinol (THC).

Additionally, certain conventional monitoring systems may be difficult or cumbersome for the monitoring agent to use. Further, certain conventional monitoring systems may not provide sufficient levels of information to the monitoring agent. For example, certain conventional monitoring systems may not provide a notice or warning to a monitoring agent that they are in close proximity to the target individual.

### SUMMARY

In one aspect, some embodiments of a method of testing a user for a substance include connecting a portable testing device to a personal mobile device; recording a video from a camera via the personal mobile device; capturing a first photograph from the camera via the personal mobile device; measuring a blood alcohol content of the user via the portable testing device while recording the video via the personal mobile device; capturing a second photograph from the camera via the personal mobile device; comparing the first photograph and the second photograph via a facial recognition routine; analyzing the blood alcohol content of the user compared to a threshold blood alcohol content; uploading at least one of the video, the first photograph, and the second photograph to a remote server; and generating a report based on the analyzing the blood alcohol content of the user compared to the threshold blood alcohol content

In one aspect, some embodiments of a method of testing a user for a substance include connecting a portable testing device to a personal mobile device via wireless communication; activating a camera of the personal mobile device; providing a video preview of the camera to the user via the personal mobile device; recording a video from the camera via the personal mobile device; capturing a first photograph from the camera via the personal mobile device; measuring a blood alcohol content of the user via the portable testing device while recording the video via the personal mobile device; capturing a second photograph from the camera via the personal mobile device; capturing a third photograph via a testing camera of the portable testing device; comparing the first photograph and the second photograph via a facial recognition routine; analyzing the blood alcohol content of the user compared to a threshold blood alcohol content; analyzing a suction airflow of the user after analyzing the blood alcohol content of the user; recording a position of at least the portable testing device or a position of the personal mobile device; uploading at least one of the video, the first photograph, and the second photograph to a remote server; and generating a report based on the analyzing the blood alcohol content of the user compared to the threshold blood alcohol content.

### BRIEF DESCRIPTION OF DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1A depicts a perspective view of a portable testing device.

FIG. 1B depicts a reverse perspective view of the portable testing device of FIG. 1A.

FIG. 1C depicts a front elevation view of the portable testing device of FIG. 1A.

FIG. 1D depicts a rear elevation view of the portable testing device of FIG. 1A.

FIG. 1E depicts a bottom elevation view of the portable testing device of FIG. 1A.

FIG. 2 depicts the portable testing device with a personal mobile device.

FIG. 3 depicts a method for testing a user for a substance.

FIG. 4 depicts a method for testing a user for a substance.

FIG. 5 depicts a method to confirm a vehicle restriction for a user.

FIG. 6 depicts an automotive testing device.

FIG. 7 depicts a method for testing a user for a substance in a vehicle.

FIG. 8 depicts the automotive testing device with the portable testing device.

FIG. 9 depicts another embodiment of an automotive testing device with the portable testing device.

FIG. 10 depicts a method for testing a user for a substance in a vehicle.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced using one or more implementations. In one or more instances, well-known structures and components are shown in block diagram form in order to avoid obscuring the concepts of the subject technology.

The present description relates in general to monitoring systems, and more particularly to, for example, without limitation remote sobriety monitoring systems. For the purposes of the present description, the monitoring systems will be described with respect to remote sobriety monitoring systems for probation and conditional release applications e.g. court ordered programs. However, the various embodiments of the monitoring systems are not limited to the aforementioned applications, but to any conceivable application of remote monitoring systems.

Embodiments described herein can provide a monitoring system that is more difficult to defeat or bypass by the target individual. As described herein, embodiments can include verification steps that prevent a device or third party from performing a required breathalyzer test for the target individual. Further, embodiments described herein can provide reminder to the target individual to perform a scheduled breathalyzer test. Additionally, embodiments described herein can identify if the target individual is utilizing a vehicle without a required vehicle interlock device. Further, embodiments described herein can provide a streamlined interface for the monitoring agent.

Various aspects of the present disclosure provide a remote monitoring systems that includes one or more hardware devices, an application for use by the target individual, an application for use by the monitoring agent, and a centralized service to facilitate communication between the components of the remote monitoring system. In some embodiments, the hardware devices can include, but are not limited to a handheld breathalyzer and/or a vehicle interlock device. In some embodiments, the application for use by the target individual can be an application installed on a user's mobile phone, tablet, laptop, or other personal computing device. Optionally, the hardware devices can be linked or otherwise connected to the user's personal computing device via a wireless connection such as Wi-Fi, Bluetooth, or other local or personal area network. In some embodiments, a user's device may notify the user if the phone is out of communication range with the hardware device(s) of the remote monitoring system. The user's device may report that the hardware device(s) was out of range and the amount of time the device(s) was out of range. In some embodiments, the application for use by the monitoring agent can be an application installed on a user's mobile phone, tablet, laptop, or other personal computing device. In some embodiments, the centralized service that facilitates communication between the components of the remote monitoring system can be server based, cloud based, or hosted on the computing device or the target user or the monitoring agent.

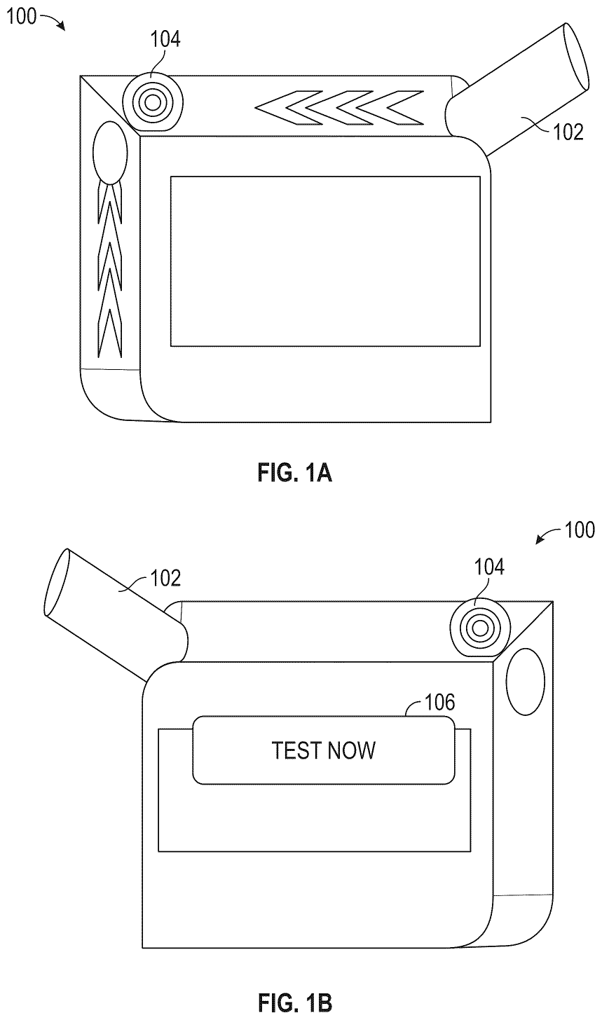

FIG. 1A depicts a perspective view of a portable testing device. FIG. 1B depicts a reverse perspective view of the portable testing device of FIG. 1A. FIG. 1C depicts a front elevation view of the portable testing device of FIG. 1A. FIG. 1D depicts a rear elevation view of the portable testing device of FIG. 1A. FIG. 1E depicts a bottom elevation view of the portable testing device of FIG. 1A. With reference to FIGS. 1A-1E, the remote monitoring system can include a handheld device or portable testing device 100 for use by the target individual. The portable testing device 100 can allow the target individual to perform sobriety tests (e.g. breathalyzer tests) at required times. In the depicted example, the portable testing device 100 includes a fuel cell sensor and pump to facilitate the measurement of blood alcohol content of a user. As illustrated, the portable testing device 100 includes a mouthpiece 102 to allow a user to blow (or provide suction) onto the fuel cell sensor of the portable testing device 100.

In some embodiments, the portable testing device 100 can include a global positioning system (GPS) receiver to receive location coordinates. In some applications, the position of the portable testing device 100 can be monitored before, during, and after testing. The portable testing device 100 can include onboard memory to store test and position information. Further, the portable testing device 100 can include a battery and one or more wireless radios.

In the depicted example, the portable testing device 100 can include a camera 104. In some applications, the camera 104 can be used to record photographs or videos of the user before, during, and after testing. Media from the camera 104 can be stored onboard or provided to an attached device via the wireless radio.

In some embodiments, the portable testing device 100 includes a display 106 to communicate status and prompts to the user. For example, the display 106 can provide instructions or readiness information to the user.

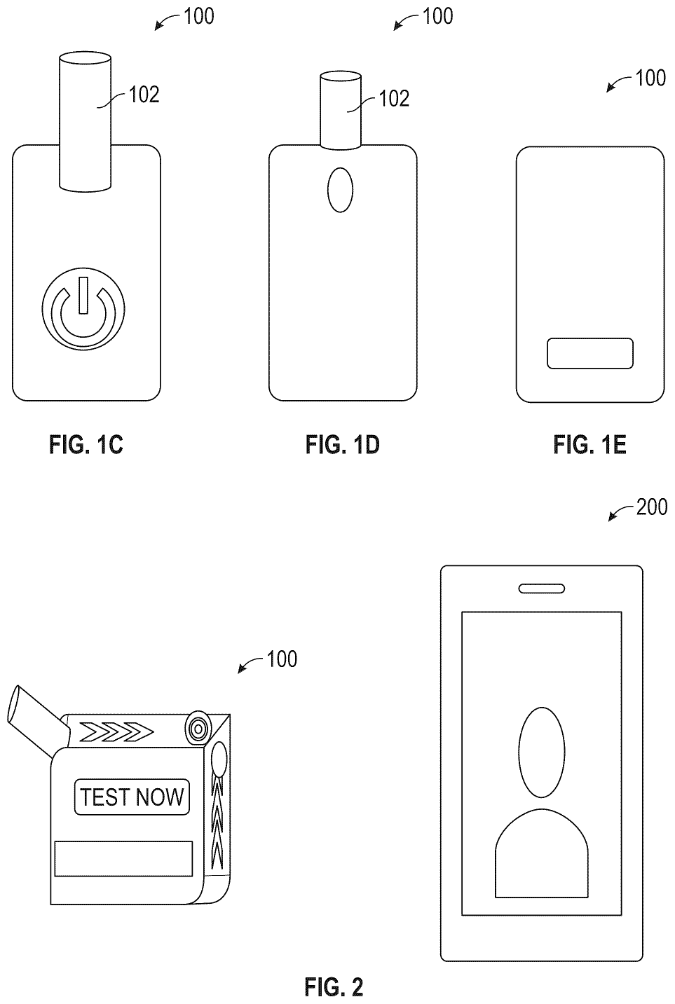

FIG. 2 depicts the portable testing device 100 with a personal mobile device 200. In the depicted example, the portable testing device 100 can be used in conjunction with a companion application executed on a user's phone or other personal mobile device 200. In some embodiments, the companion application captures photographs through the personal mobile device 200 before and after testing. In some applications, the personal mobile device 200 further records video while the target individual submits a test via the portable testing device 100. In some embodiments, the camera 104 of the portable testing device 100 can provide a close-up photo to provide biometric recognition of the test subject. Optionally, the portable testing device 100 can require the target individual to inhale through the handheld device after the test to confirm a human breath was provided to the portable testing device 100. In some embodiments, if a test is not performed in an allotted time window, a missed test alert along with the GPS coordinates of the portable testing device 100 can be provided to the centralized management service. Optionally, the results and/or status of testing is disclosed to the monitoring agent without disclosing to the target individual.

Since a user's personal mobile device 200 can be tethered or otherwise connected to the portable testing device 100, the portable testing device 100 can monitor the location of the personal mobile device 200. Therefore, if the personal mobile device 200 exceeds a threshold distance (e.g. 50 feet) from the portable testing device 100, the portable testing device 100 can notify the user. Similarly, in some embodiments, the personal mobile device 200 can monitor the location of the portable testing device 100. Therefore, if the portable testing device 100 exceeds a threshold distance from the personal mobile device 200, the personal mobile device 200 can notify the user. In some applications, if the two devices are out of range from each other for more than an allotted period of time (e.g. 1 hour), the out of range event can be logged and flagged for review by the monitoring agency.

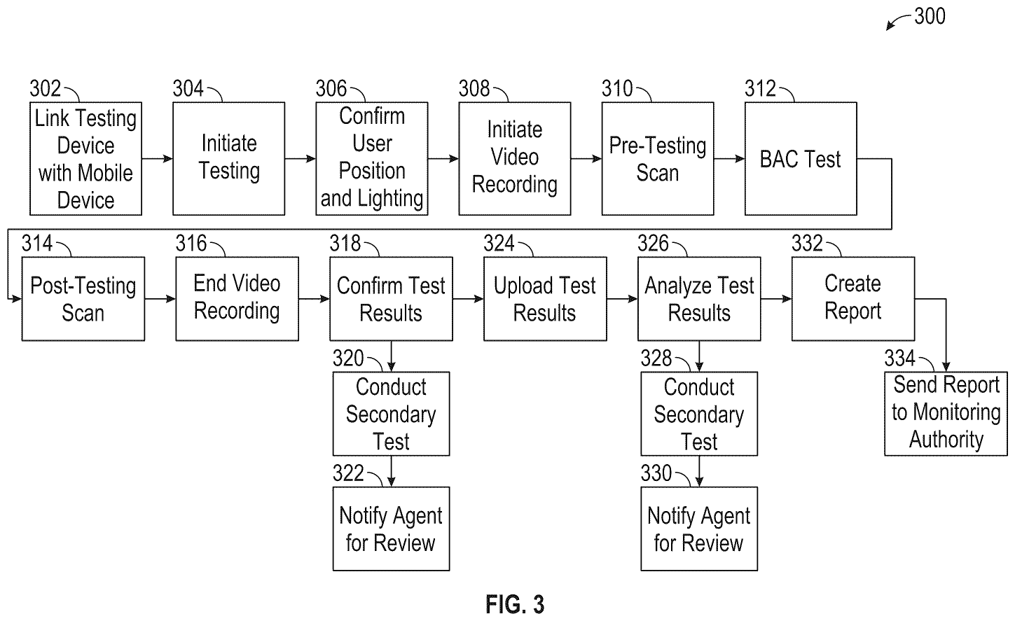

FIG. 3 depicts a method 300 for testing a user for a substance. In step 302, the portable testing device is linked with a personal or mobile device. In the depicted example, the portable testing device can be linked to a mobile device via a wireless connection such as Wi-Fi, Bluetooth, or other local or personal area network.

In step 304, testing of the user can be initiated. Testing can be initiated by the user or can be prompted by the user's mobile device. In some embodiments, a user may be required to perform testing either on a schedule, randomly, or when prompted by the user's companion application or by a supervising officer. In some applications, if a user does not perform or otherwise submit to testing in an allotted period of time, a missed test report can be provided to the monitoring agent. In some embodiments, GPS coordinates at the time of missed test is provided.

In step 306, a video preview of the user's mobile device camera can be provided. In preparation for testing, the video preview can allow the user to confirm lighting conditions, camera positioning, and other environmental factors to allow for a successful testing process and minimize retesting. After the video preview is provided, video recording and testing can be initiated by the user in step 308. In the depicted example, video is recorded on the user's mobile device concurrently during the testing process.

In step 310, a pre-testing photograph or scan can be taken of the user. The pre-testing scan can be taken by the mobile device or the testing device. In step 312, the user can blow into the testing device, or otherwise perform the breathalyzer test, while the mobile device is recording the user. In some embodiments, the user will be required to inhale or otherwise provide suction to the testing device to confirm or verify a human input. After performing the breathalyzer test, in step 314 a post-testing photograph or scan is taken of the user. Similarly, the post-testing scan be taken by the mobile device or the testing device. In step 316, the video recording of the user is stopped.

In some embodiments, in step 318, the pre-testing scan and the post-testing scan of the user are compared to confirm the validity of the test. In some applications, facial recognition software or algorithms are implemented to determine if the test subject is the intended monitoring subject or if the testing was otherwise tampered. Further, additional processing (e.g. feasibility analysis) may be performed to determine if the test results are conclusive or otherwise acceptable. If the test results are inconclusive, the user may be prompted to conduct a secondary test in step 320. In some applications, the monitoring agent can be notified for further review of the inconclusive test results in step 322.

In step 324, confirmed or conclusive test results can be uploaded to a centralized service via a server or cloud provider. In step 326, the test results can be analyzed. In some applications, the user's blood alcohol content is compared to a preset threshold value. If the user's blood alcohol content exceeds the threshold or if the test is otherwise inconclusive, the user is prompted to conduct a secondary or confirmatory test in step 328. Further, in some applications, the monitoring agent can be notified for further review of the test results in step 330.

In step 332, reports based on the test results and the recorded media are generated. The test reports can be centrally generated and/or stored. In step 334 the reports are sent to monitoring agents and/or officers. As described herein, test results, media, and other data can be accessed through agent and/or officer portals.

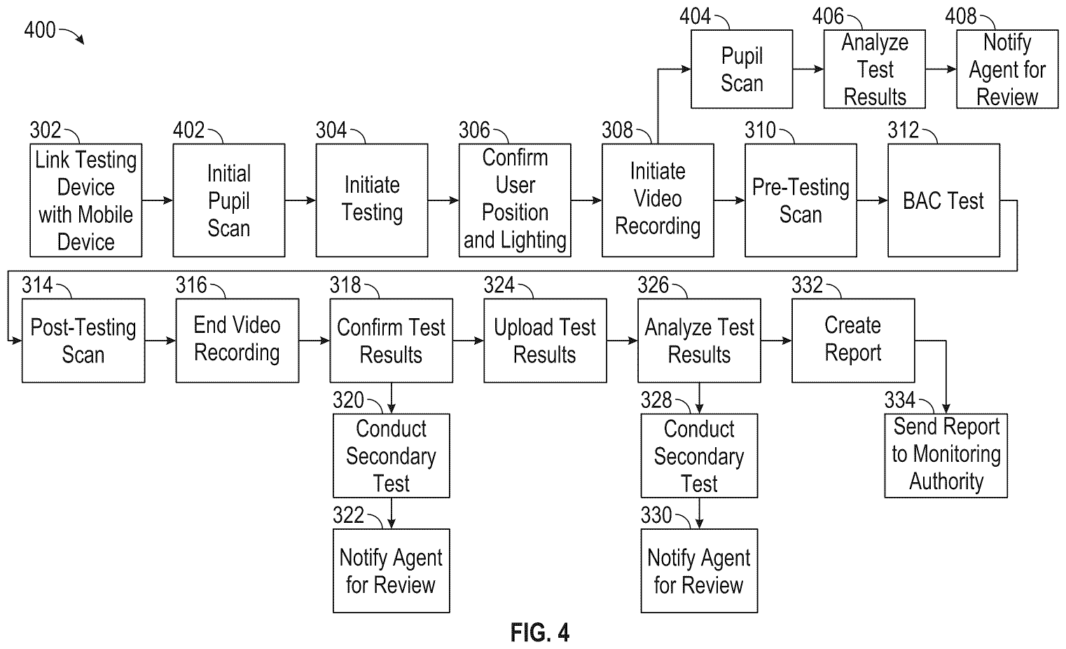

FIG. 4 depicts a method 400 for testing a user for a substance. In some applications, a testing process or method may include testing for additional substances, such as THC. As illustrated, a testing process may be similar to method 300 but include additional steps to monitor for THC consumption. In step 402, a baseline or initial pupil scan or photograph can be taken. In some applications, the baseline photo is taken during a user orientation or other controlled setting.

In step 404, during the testing process, a photograph or scan of the user's pupil can be taken. In step 406, the photo of a target individual's retina or pupil can be analyzed to determine THC consumption. For example, if pupil dilation exceeds 25% the test can be flagged and the monitoring agent can be alerted in step 408. Optionally, the THC consumption test can be performed by the camera of the handheld device or by a camera of a user's smartphone or other computing device. In some embodiments, the THC consumption test can be performed concurrently during a breathalyzer test. In some embodiments, certain steps of the monitoring process may be introduced, optional, omitted, repeated, or modified.

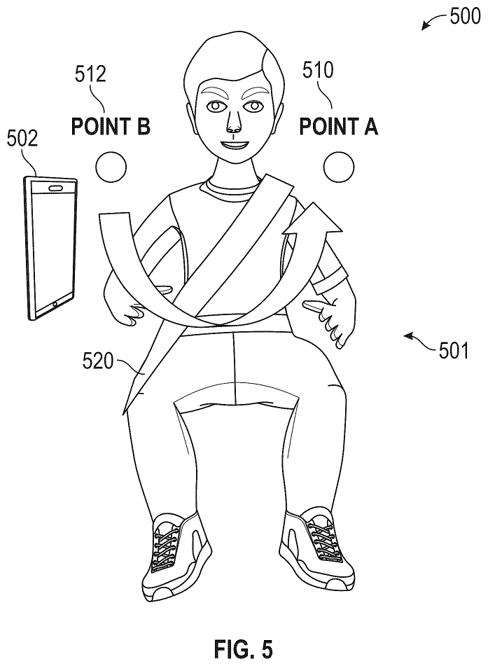

FIG. 5 depicts a method to confirm a vehicle restriction for a user. In some applications, the testing device and/or a user's mobile device 502 can be used to confirm that the target individual 501 is not operating a motor vehicle a restricted vehicle (i.e. a vehicle that does not include a vehicle interlock device). In some embodiments, the handheld device or the user's mobile device 502 can detect if the vehicle interlock device was used and if the target individual 501 is traveling at a rate of speed (e.g. 30 mph) that suggests that the target individual 501 is in a vehicle. In response to detection of the use of the vehicle interlock device and the rate of speed, the application may request photographic proof and/or a sobriety test to confirm that the individual is not driving. The remote monitoring system may require the target individual 501 to submit a video confirming the individual is not driving an unauthorized vehicle. In some embodiments, the target individual 501 can be instructed to take a video from a first point A 510 to a second point B 512, illustrating the orientation of their seat belt 520 and their surroundings to confirm they not driving an unauthorized vehicle. The target individual 501 may be required to perform follow up tests. In some embodiments, if the target individual 501 does not provide the required video or test in the prescribed time period, a report may be provided to the monitoring agent. In some embodiments, certain steps of the monitoring process may be introduced, optional, omitted, repeated, or modified.

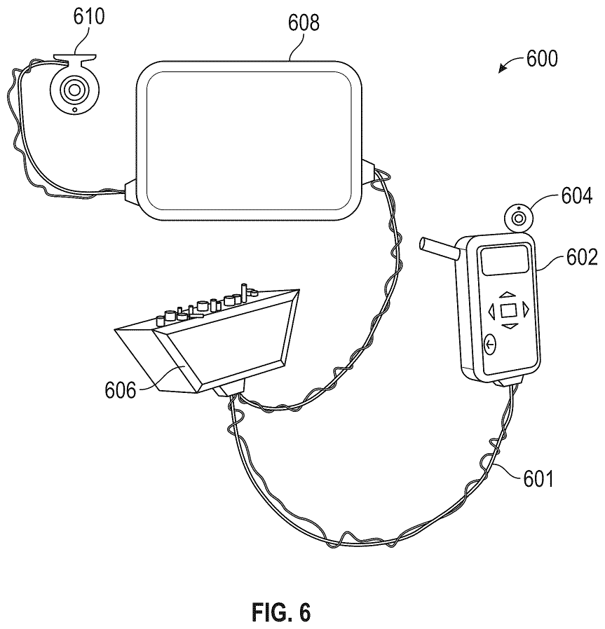

FIG. 6 depicts an automotive testing device. In some embodiments, the remote monitoring system can include an automotive testing device or vehicle interlock device 600. As illustrated, the vehicle interlock device 600 can include a handheld breathalyzer 602, a camera component 604, a control center 608, and an On-Board Diagnostics (OBD) connector 606. In some embodiments, the components of the vehicle interlock device 600 are connected via one or more cables 601. In the depicted example, the breathalyzer 602 can include a camera 604 that is configured to capture a photo of the driver during use of the breathalyzer 602. Further, a camera component 610 can be mounted to the vehicle and can capture photographs during an initial start test, a rolling retest, a missed rolling retest, and upon an engine off event. Photographs of the driver from the camera component 610 and the integrated camera 604 of the breathalyzer 602 can be compared to validate the test subject. In the event of a mismatch, the test can be flagged and the monitoring agent can be notified after confirmation. The control center 608 can include a GPS receiver to record the location of any tests and the drive route. The control center 608 can draw power from the vehicle's OBD port and interface with the vehicle to prevent unauthorized operation of the vehicle. In some embodiments, certain steps of the monitoring process may be introduced, optional, omitted, repeated, or modified.

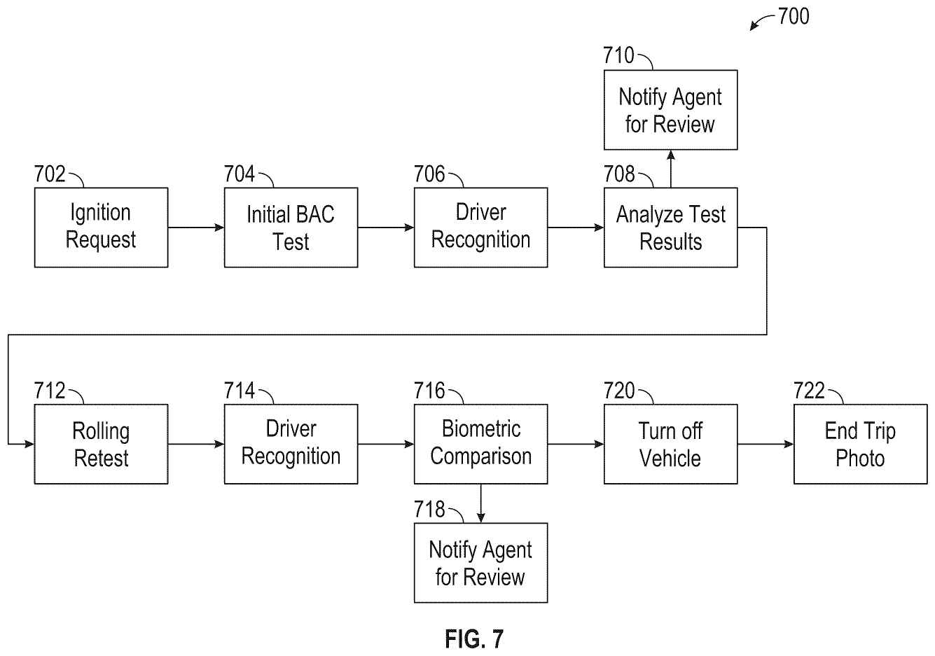

FIG. 7 depicts a method 700 for testing a user for a substance in a vehicle. In some applications, the method 700 can utilize the vehicle interlock device 600 described above. In step 702, the user requests ignition (or starting) of the vehicle to initiate the interlock process. The user may request operation of the vehicle by turning a key, pressing a start button, or any other method to start or request starting of the vehicle. In step 704, upon requesting ignition or starting of the vehicle, the driver of the vehicle must submit to a breathalyzer or blood alcohol content test. In step 706, the driver monitoring system can utilize one or more cameras to recognize the driver (or test subject) either simultaneously or immediate after the blood alcohol test process. In some applications, the driver monitoring system can compare a photograph from a windshield mounted camera and a photograph from the breathalyzer device to determine if the test subject is the driver. In some applications, the driver monitoring system can utilize facial recognition algorithms or software to identify the test subject.

In step 708, the test results can be analyzed. In some applications, the user's blood alcohol content is compared to a preset threshold value. If a user's blood alcohol content is below the predefined threshold, operation of the vehicle is permitted. If the user's blood alcohol content exceeds the threshold or if the test is otherwise inconclusive, the user is denied operation of the vehicle and may be prompted to conduct a secondary or confirmatory test. Further, additional processing (e.g. feasibility analysis) may be performed to determine if the test results are conclusive or otherwise acceptable. If the test results are inconclusive, the user may be prompted to conduct a secondary test. Further, in some applications, the monitoring agent can be notified for further review of the test results in step 710.

During operation of the vehicle, the driver may be required to submit to rolling retests in step 712. During a rolling retest, the driver of the vehicle must submit to a breathalyzer or blood alcohol content test during periodic or random intervals during operation of the vehicle. Similarly, during step 716, the driver monitoring system can utilize one or more cameras to recognize the driver (or test subject) either simultaneously or immediate after the rolling retest procedure. In some applications, the driver monitoring system can compare a photograph from a windshield mounted camera and a photograph from the breathalyzer device to determine if the test subject is the driver (and the initial test subject). In some applications, the driver monitoring system can utilize facial recognition algorithms or software to identify the test subject.

After the rolling retest, the test results can be analyzed. If a user's blood alcohol content is below the predefined threshold, continued operation of the vehicle is permitted. If the user's blood alcohol content exceeds the threshold or if the test is otherwise inconclusive, the user may be prompted to conduct a secondary or confirmatory test. Further, additional processing (e.g. feasibility analysis) may be performed to determine if the test results are conclusive or otherwise acceptable. If the test results are inconclusive, the user may be prompted to conduct a secondary test. Further, in some applications, the monitoring agent can be notified for further review of the test results in step 718.

At the end of the trip, vehicle is shut off in step 720. At the end of a vehicle trip, an end trip photo is recorded in step 722. The end trip photo can be uploaded to a centralized server or cloud to be included with reporting.

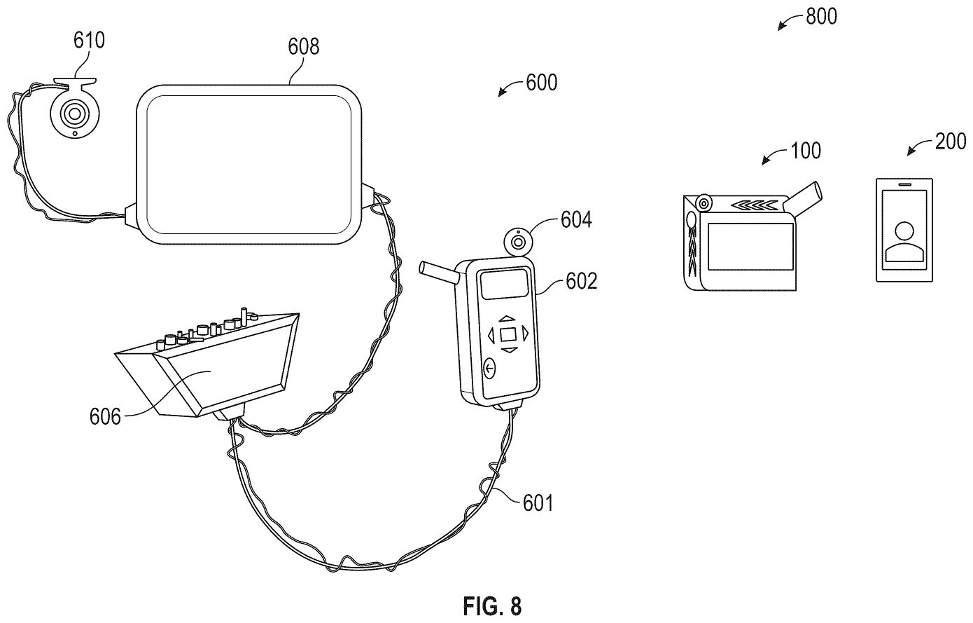

FIG. 8 depicts a monitoring system 800 that includes a vehicle interlock device 600 with a handheld device or portable testing device 100. In some applications, an automotive testing device or vehicle interlock device 600 and a handheld device 100 can be used separately or concurrently with the remote monitoring system 800. The handheld device 100 can be configured to sleep or otherwise disengage when the vehicle interlock device 600 registered to the target individual is activated. In some embodiments, the handheld device 100 communicates with control center 608 of the vehicle interlock device 600 to determine operational status. The handheld device 100 can communicate via wireless protocols, such as Bluetooth, Wi-Fi or other personal area networks.

Monitoring and testing via the handheld device 100 can resume once the vehicle is shut off. Data can be shared between the handheld device 100, the vehicle interlock device 600, the target individual software, and the agent software via the centralized service. As described herein, the monitoring system 800 can further incorporate the use of and communicate with a user's personal mobile device 200.

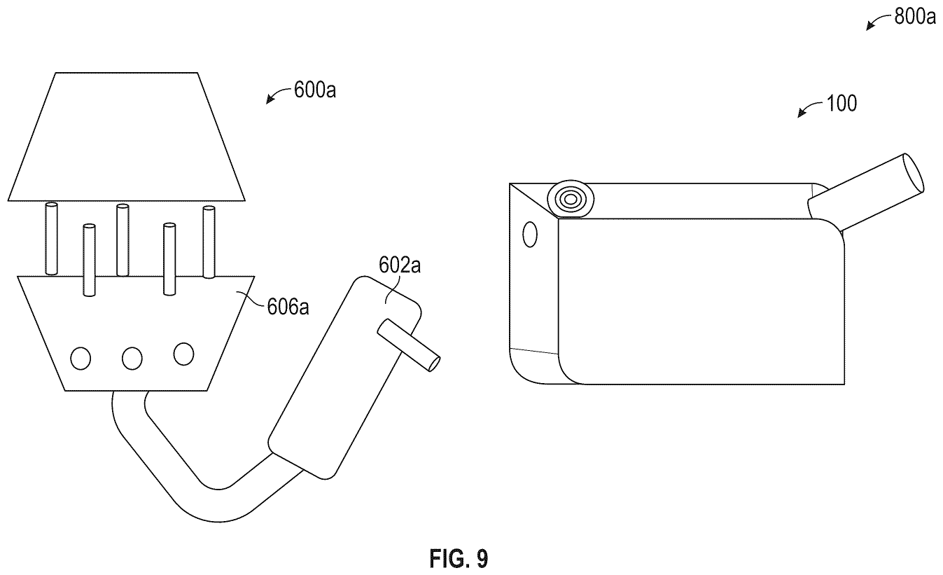

FIG. 9 depicts another embodiment of a monitoring system 800a that includes an automotive testing device 600a with the portable testing device 100. In some embodiments, the automotive testing device 600a can include a breathalyzer 602a that directly interfaces with a vehicle OBD port via plug 606a. In certain embodiments, components of the vehicle interlock device 600 described herein can be integrated directly into the breathalyzer 602a and/or the plug 606a. As illustrated, the portable testing device 100 and/or user's personal mobile device 200 can interface with the automotive testing device 600a.

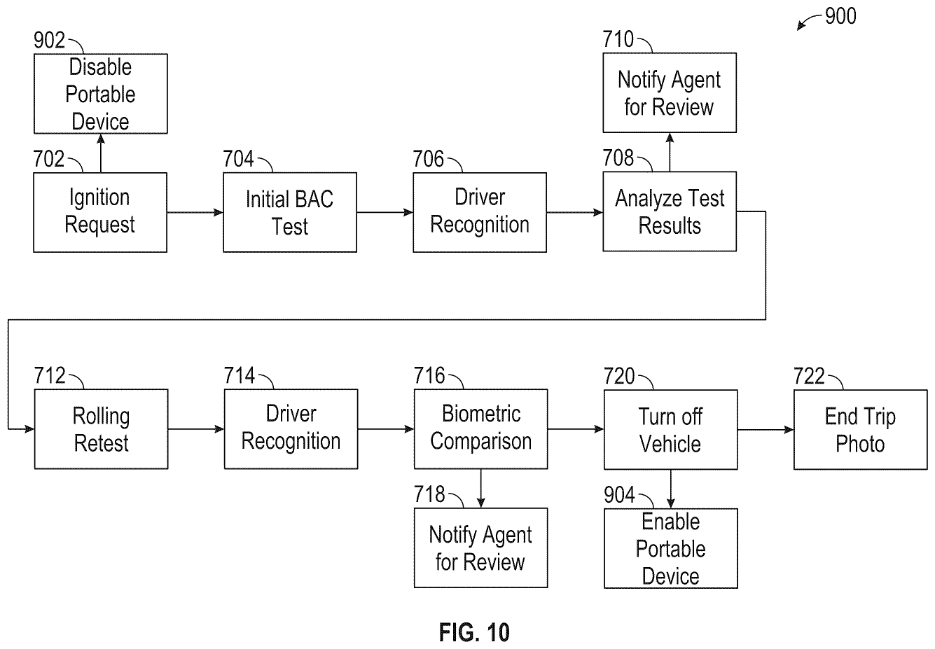

FIG. 10 depicts a method 900 for testing a user for a substance in a vehicle. In some applications, a vehicle interlock system can coordinate with a connected handheld device and/or mobile device to provide comprehensive in-vehicle and out of vehicle monitoring of the test subject. As illustrated, an interlock and testing process may be similar to method 700 but may include additional steps to facilitate interoperability and hand-offs between a handheld device and the vehicle-based system.

As illustrated in step 902, after an ignition request in step 702, the interlock system can communicate (either locally or via cloud service) with the handheld device to confirm that the user has entered and is operating an approved interlock equipped vehicle. After confirming that the user is intending to operate a registered and interlock equipped vehicle, the handheld device can be temporarily disabled (e.g. sleep mode) during vehicle operation. In some embodiments, the handoff between the handheld device and the vehicle interlock can be logged.

Similarly, after the vehicle is turned off in step 720, in step 904, the interlock system can communicate (either locally or via cloud service) with the handheld device to confirm that the vehicle trip has ended. After confirming that the user has ended the trip, the handheld device can be re-enabled for continued operation. In some embodiments, the handoff between the vehicle interlock and the handheld device can be logged.

In some applications, the remote monitoring system can alert an agent or officer that a target or monitored individual is within a certain radius of the agent. For example, an agent may receive an alert when the target individual is within 500 feet during a specific time of day (e.g. outside of work hours). Optionally, the agent may request an on-demand test of the target individual. Further, the remote monitoring system can provide emergency functionality, such as calling emergency services, sending GPS coordinates, and/or recording video.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A method of testing a user for a substance, the method including: connecting a portable testing device to a personal mobile device; recording a video from a camera via the personal mobile device; capturing a first photograph from the camera via the personal mobile device; measuring a blood alcohol content of the user via the portable testing device while recording the video via the personal mobile device; capturing a second photograph from the camera via the personal mobile device; comparing the first photograph and the second photograph via a facial recognition routine; analyzing the blood alcohol content of the user compared to a threshold blood alcohol content; uploading at least one of the video, the first photograph, and the second photograph to a remote server; and generating a report based on the analyzing the blood alcohol content of the user compared to the threshold blood alcohol content.

Clause 2. The method of Clause 1, further including: activating the camera of the personal mobile device; and providing a video preview of the camera to the user via the personal mobile device.

Clause 3. The method of Clause 1, further including: capturing a third photograph via a testing camera of the portable testing device.

Clause 4. The method of Clause 1, further including: analyzing a suction airflow of the user after analyzing the blood alcohol content of the user.

Clause 5. The method of Clause 4, further including: prompting the user to retest in response to inconclusive confirmation of the suction airflow.

Clause 6. The method of Clause 1, further including: prompting the user to retest in response to inconclusive measurement of the blood alcohol content of the user.

Clause 7. The method of Clause 6, further including: notifying an agent in response to inconclusive measurement of the blood alcohol content of the user.

Clause 8. The method of Clause 1, further including: prompting the user to retest in response to inconclusive comparison of the first photograph to the second photograph.

Clause 9. The method of Clause 8, further including: notifying an agent in response to inconclusive comparison of the first photograph to the second photograph.

Clause 10. The method of Clause 1, further including: prompting the user to initiate a test via the personal mobile device.

Clause 11. The method of Clause 10, further including: logging an incomplete test in response to the measuring the blood alcohol content of the user in a predetermined time.

Clause 12. The method of Clause 1, further including: measuring the blood alcohol content of the user via a fuel cell of the portable testing device.

Clause 13. The method of Clause 1, further including: connecting the portable testing device to the personal mobile device via wireless communication.

Clause 14. The method of Clause 1, further including: recording a position of at least the portable testing device or a position of the personal mobile device.

Clause 15. The method of Clause 14, further including: comparing the position of the portable testing device to the position of the personal mobile device.

Clause 16. The method of Clause 15, further including: notifying the user via the portable testing device in response to the position of the portable testing device being a threshold distance away from the position of the personal mobile device.

Clause 17. The method of Clause 15, further including: notifying the user via the personal mobile device in response to the position of the personal mobile device being a threshold distance away from the position of the portable testing device.

Clause 18. A method of testing a user for a substance, the method including: connecting a portable testing device to a personal mobile device via wireless communication; activating a camera of the personal mobile device; providing a video preview of the camera to the user via the personal mobile device; recording a video from the camera via the personal mobile device; capturing a first photograph from the camera via the personal mobile device; measuring a blood alcohol content of the user via the portable testing device while recording the video via the personal mobile device; capturing a second photograph from the camera via the personal mobile device; capturing a third photograph via a testing camera of the portable testing device; comparing the first photograph and the second photograph via a facial recognition routine; analyzing the blood alcohol content of the user compared to a threshold blood alcohol content; analyzing a suction airflow of the user after analyzing the blood alcohol content of the user; recording a position of at least the portable testing device or a position of the personal mobile device; uploading at least one of the video, the first photograph, and the second photograph to a remote server; and generating a report based on the analyzing the blood alcohol content of the user compared to the threshold blood alcohol content.

Clause 19. The method of Clause 18, further including: prompting the user to retest in response to inconclusive confirmation of measurement of the blood alcohol content of the user, comparison of the first photograph to the second photograph, or the suction airflow.

Clause 20. The method of Clause 18, further including: notifying an agent in response to inconclusive measurement in response to inconclusive confirmation of measurement of the blood alcohol content of the user, comparison of the first photograph to the second photograph, or the suction airflow.

### Further Considerations

Any spatial references, such as, for example, “upper,” “lower,” “above,” “below,” “between,” “bottom,” “vertical,” “horizontal,” “angular,” “upwards,” “downwards,” “side-to-side,” “left-to-right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In several exemplary embodiments, while different steps, processes, and procedures are described as appearing as distinct acts, one or more of the steps, one or more of the processes, and/or one or more of the procedures may also be performed in different orders, simultaneously and/or sequentially. In several exemplary embodiments, the steps, processes, and/or procedures may be merged into one or more steps, processes and/or procedures.

In several exemplary embodiments, one or more of the operational steps in each embodiment may be omitted. Moreover, in some instances, some features of the present disclosure may be employed without a corresponding use of the other features. Moreover, one or more of the above-described embodiments and/or variations may be combined in whole or in part with any one or more of the other above-described embodiments and/or variations.

Although several exemplary embodiments have been described in detail above, the embodiments described are exemplary only and are not limiting, and those skilled in the art will readily appreciate that many other modifications, changes and/or substitutions are possible in the exemplary embodiments without materially departing from the novel teachings and advantages of the present disclosure. Accordingly, all such modifications, changes, and/or substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112, paragraph 6 for any limitations of any of the claims herein, except for those in which the claim expressly uses the word “means” together with an associated function.

1. A method of testing a user for a substance, the method comprising:
connecting a portable testing device to a personal mobile device;
recording a video from a camera via the personal mobile device;
capturing a first photograph from the camera via the personal mobile device;
measuring a blood alcohol content of the user via the portable testing device while recording the video via the personal mobile device;
capturing a second photograph from the camera via the personal mobile device;
comparing the first photograph and the second photograph via a facial recognition routine;
analyzing the blood alcohol content of the user compared to a threshold blood alcohol content;
uploading at least one of the video, the first photograph, and the second photograph to a remote server; and
generating a report based on the analyzing the blood alcohol content of the user compared to the threshold blood alcohol content.

2. The method of claim 1, further comprising:
activating the camera of the personal mobile device; and
providing a video preview of the camera to the user via the personal mobile device.

3. The method of claim 1, further comprising:
capturing a third photograph via a testing camera of the portable testing device.

4. The method of claim 1, further comprising:
analyzing a suction airflow of the user after analyzing the blood alcohol content of the user.

5. The method of claim 4, further comprising:
prompting the user to retest in response to inconclusive confirmation of the suction airflow.

6. The method of claim 1, further comprising:
prompting the user to retest in response to inconclusive measurement of the blood alcohol content of the user.

7. The method of claim 6, further comprising:
notifying an agent in response to inconclusive measurement of the blood alcohol content of the user.

8. The method of claim 1, further comprising:
prompting the user to retest in response to inconclusive comparison of the first photograph to the second photograph.

9. The method of claim 8, further comprising:
notifying an agent in response to inconclusive comparison of the first photograph to the second photograph.

10. The method of claim 1, further comprising:
prompting the user to initiate a test via the personal mobile device.

11. The method of claim 10, further comprising:
logging an incomplete test in response to the measuring the blood alcohol content of the user in a predetermined time.

12. The method of claim 1, further comprising:
measuring the blood alcohol content of the user via a fuel cell of the portable testing device.

13. The method of claim 1, further comprising:
connecting the portable testing device to the personal mobile device via wireless communication.

14. The method of claim 1, further comprising:
recording a position of at least the portable testing device or a position of the personal mobile device.

15. The method of claim 14, further comprising:
comparing the position of the portable testing device to the position of the personal mobile device.

16. The method of claim 15, further comprising:
notifying the user via the portable testing device in response to the position of the portable testing device being a threshold distance away from the position of the personal mobile device.

17. The method of claim 15, further comprising:
notifying the user via the personal mobile device in response to the position of the personal mobile device being a threshold distance away from the position of the portable testing device.

18. A method of testing a user for a substance, the method comprising:
connecting a portable testing device to a personal mobile device via wireless communication;
activating a camera of the personal mobile device;
providing a video preview of the camera to the user via the personal mobile device;
recording a video from the camera via the personal mobile device;
capturing a first photograph from the camera via the personal mobile device;
measuring a blood alcohol content of the user via the portable testing device while recording the video via the personal mobile device;
capturing a second photograph from the camera via the personal mobile device;
capturing a third photograph via a testing camera of the portable testing device;
comparing the first photograph and the second photograph via a facial recognition routine;
analyzing the blood alcohol content of the user compared to a threshold blood alcohol content;
analyzing a suction airflow of the user after analyzing the blood alcohol content of the user;
recording a position of at least the portable testing device or a position of the personal mobile device;
uploading at least one of the video, the first photograph, and the second photograph to a remote server; and
generating a report based on the analyzing the blood alcohol content of the user compared to the threshold blood alcohol content.

19. The method of claim 18, further comprising:
prompting the user to retest in response to inconclusive confirmation of measurement of the blood alcohol content of the user, comparison of the first photograph to the second photograph, or the suction airflow.

20. The method of claim 18, further comprising:
notifying an agent in response to inconclusive measurement in response to inconclusive confirmation of measurement of the blood alcohol content of the user, comparison of the first photograph to the second photograph, or the suction airflow.
